(19) United States
(12) Patent Application Publication
(10) Pub. No.: US 2020/0107926 A1
(43) Pub. Date: Apr. 9, 2020

(54) METHOD OF BIOLOGIC ACROMIAL RESURFACING

(71) Applicant: Arthrex, Inc., Naples, FL (US)

(72) Inventors: Thomas Dooney, JR., Naples, FL (US); Matthew Ravenscroft, Mere Cheshire (GB)

(21) Appl. No.: 16/153,061

(22) Filed: Oct. 5, 2018

(51) Int. Cl. A61F 2/08; A61B 17/04

(52) U.S. Cl. CPC A61F 2/0811; A61B 17/0469; A61B 17/0467; A61F 2002/087; A61F 2230/0019; A61F 2002/0852; A61F 2240/002

(57) ABSTRACT

A graft is prepared for attachment to an acromion. Two sutures are attached to corners of the graft. The two sutures are loaded into an introducer instrument, and the introducer instrument spreads the graft open. The graft is then positioned under the acromion. Strands of like sutures are tied on a medial side of the acromion to attach the graft to the acromion. The sutures are trimmed after knotting, securing the graft to the acromion.

### BACKGROUND

This disclosure relates to a method of biologic acromial resurfacing.

### SUMMARY

A graft is attached to an acromion of a scapula to resurface the acromion and act as padding between a humerus and the acromion. A method also relates to introducing and fixing the graft to the acromion.

Embodiments of a method of preparing a graft for attachment to an acromion of a scapula are disclosed herein. A graft is first prepared. A surface area of the acromion is measured, and the graft is trimmed to an appropriate size. A suture loop is passed through a corner of the graft, and a tail of a first suture is fed through the loop and tightened (this technique is called a “Lasso Loop”). The first suture is attached to the opposite corner of the graft in the same manner. A second suture is attached to the other two corners in the same manner. The two sutures attached to the graft spread the graft open relative to the acromion.

The graft is then delivered to the acromion. The two sutures are loaded into an introducer instrument, and the instrument spreads the graft to be located under the acromion. The tails of like sutures are tied on a medial side of the acromion to attach the graft to the acromion. The sutures are trimmed after knotting, securing the graft to the acromion.

Embodiments of a method of resurfacing an acromion disclosed herein include attaching at least one suture to a graft and securing the graft to an acromion with the at least one suture.

In an embodiment, the method includes measuring a surface area of the acromion and trimming the graft to fit the acromion based on a measurement.

In an embodiment, at least one suture includes a first suture and a second suture, the method including loading the first suture and the second suture into an introducer instrument that spreads the graft to be substantially flat and positioning the graft under the acromion.

In an embodiment, the graft can substantially rectangular in shape and includes a posterior/medial corner, an anterior/medial corner, a posterior/lateral corner, and an anterior/lateral corner, wherein the first suture is secured to the posterior/lateral corner and the anterior/medial corner such that a posterior/lateral tail extends from the posterior/lateral corner and an anterior/medial tail extends from the anterior/medial corner, wherein the second suture is secured to the posterior/medial corner and the anterior/lateral corner such that a posterior/medial tail extends from the posterior/medial corner and an anterior/lateral tail extends from the anterior/lateral corner, and the first suture and the second suture intersect on the graft and retain the graft flat.

In an embodiment, the method includes retrieving the posterior/medial suture tail and then pulling the posterior/medial suture tail through an incision, retrieving the anterior/medial suture tail and then pulling the anterior/medial suture tail through an incision, retrieving the posterior/lateral suture tail and then pulling the posterior/lateral suture tail through an incision, and retrieving the anterior/lateral suture tail and then pulling the anterior/lateral suture tail through an incision.

In an embodiment, the method includes retrieving the posterior/lateral suture tail and the anterior/medial suture tail of the first suture through a lateral side through a medial percutaneous incision, tying the posterior/lateral suture tail and the anterior/medial suture tail in a first knot on the medial side of the acromion, retrieving the anterior/lateral suture tail and the posterior/medial suture tail of the second suture through the lateral side through the medial percutaneous incision, and tying the anterior/lateral suture tail and the posterior/medial suture tail in a second knot on the medial side of the acromion.

In an embodiment, the method includes trimming the posterior/lateral suture tail and the anterior/medial suture tail of the first knot and trimming the anterior/lateral suture tail and the posterior/medial suture tail of the second knot.

In an embodiment, the method includes including loading the posterior/medial suture tail and the anterior/medial suture tail into the introducer instrument, locating the graft under the acromion, retrieving the posterior/medial suture tail and the anterior/medial suture tail from portals, removing the introducer instrument, and retrieving the posterior/lateral suture tail and the anterior/lateral suture tail from portals

In an embodiment, the method includes wrapping the graft around the acromion, wherein the graft includes a body portion, a first end region, and a second end region including a slot, and inserting the first end region of the graft through the slot in the second end region of the graft and tensioning the graft around the acromion.

In an embodiment, the method includes stitching the first end region and the second end region together to secure the graft to the acromion.

Another embodiment of a method of resurfacing an acromion disclosed herein include attaching a first suture and a second suture to a graft, wherein the graft is substantially rectangular in shape and includes a posterior/medial corner, an anterior/medial corner, a posterior/lateral corner, and an anterior/lateral corner. The first suture is secured to the posterior/lateral corner and the anterior/medial corner such that a posterior/lateral tail extends from the posterior/lateral corner and an anterior/medial tail extends from the anterior/medial corner. The second suture is secured to the posterior/medial corner and the anterior/lateral corner such that a posterior/medial tail extends from the posterior/medial corner and an anterior/lateral tail extends from the anterior/lateral corner. The first suture and the second suture intersect on the graft and retain the graft flat. The method includes loading the first suture and the second suture into an introducer instrument that spreads the graft to be substantially flat, positioning the graft under the acromion, retrieving the posterior/medial suture tail and then pulling the posterior/medial suture tail through an incision, retrieving the anterior/medial suture tail and then pulling the anterior/medial suture tail through an incision, retrieving the posterior/lateral suture tail and then pulling the posterior/lateral suture tail through an incision, and retrieving the anterior/lateral suture tail and then pulling the anterior/lateral suture tail through an incision. The method includes retrieving the posterior/lateral suture tail and the anterior/medial suture tail of the first suture through a lateral side through a medial percutaneous incision, tying the posterior/lateral suture tail and the anterior/medial suture tail in a first knot on the medial side of the acromion, retrieving the anterior/lateral suture tail and the posterior/medial suture tail of the second suture through the lateral side through the medial percutaneous incision, and tying the anterior/lateral suture tail and the posterior/medial suture tail in a second knot on the medial side of the acromion. The method includes trimming the posterior/lateral suture tail and the anterior/medial suture tail of the first knot, and trimming the anterior/lateral suture tail and the posterior/medial suture tail of the second knot.

In an embodiment, the method includes measuring a surface area of the acromion and trimming the graft to fit the acromion based on a measurement.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1A, 1B, and 1C illustrate a schematic perspective view of a graft with a first suture and a second suture;

FIG. 2 illustrates a schematic perspective view of the graft and the sutures received in an introducer instrument;

FIG. 3 illustrates a schematic perspective view of a posterior/medial suture tail being retrieved;

FIGS. 4a illustrates a schematic perspective view of a posterior/medial suture tail being retrieved;

FIG. 4b illustrates a schematic perspective view of an anterior/medial suture tail being retrieved;

FIGS. 5a and 5b illustrate a schematic perspective view of a posterior/lateral suture tail being retrieved;

FIGS. 6a and 6b illustrate a schematic perspective view of an anterior/lateral suture tail being retrieved;

FIGS. 7a and 7b illustrate a schematic perspective view of the sutures tied around the acromion;

FIG. 8a illustrates a bottom schematic perspective view of the acromion with the graft secured by the sutures;

FIG. 8b illustrates a side schematic perspective view of the acromion with the graft secured by the sutures;

FIG. 9a illustrates a schematic perspective view of another embodiment of the graft;

FIG. 9b illustrates a schematic top view of the another embodiment of the graft; and

FIG. 9c illustrates a schematic perspective view of another embodiment of the graft attached to an acromion.

### DETAILED DESCRIPTION

A graft is attached to an acromion of a scapula to resurface the acromion and act as padding between a humerus and the acromion. A method also relates to introducing and fixing the graft to the acromion.

Embodiments of a method of preparing a graft for attachment to an acromion of a scapula are disclosed herein. A graft is first prepared. A surface area of the acromion is measured, and the graft is trimmed to an appropriate size. A suture loop is passed through a corner of the graft, and a tail of a first suture is fed through the loop and tightened (this technique is called a “Lasso Loop”). The first suture is attached to the opposite corner of the graft in the same manner. A second suture is attached to the other two corners in the same manner. The two sutures attached to the graft spread the graft open relative to the acromion.

The graft is then delivered to the acromion. The two sutures are loaded into an introducer instrument, and the instrument spreads the graft to be located under the acromion. The tails of like sutures are tied on a medial side of the acromion to attach the graft to the acromion. The sutures are trimmed after knotting, securing the graft to the acromion.

Embodiments of a method of resurfacing an acromion disclosed herein include attaching at least one suture to a graft and securing the graft to an acromion with the at least one suture.

In an embodiment, the method includes measuring a surface area of the acromion and trimming the graft to fit the acromion based on a measurement.

In an embodiment, at least one suture includes a first suture and a second suture, the method including loading the first suture and the second suture into an introducer instrument that spreads the graft to be substantially flat and positioning the graft under the acromion.

In an embodiment, the graft can substantially rectangular in shape and includes a posterior/medial corner, an anterior/medial corner, a posterior/lateral corner, and an anterior/lateral corner, wherein the first suture is secured to the posterior/lateral corner and the anterior/medial corner such that a posterior/lateral tail extends from the posterior/lateral corner and an anterior/medial tail extends from the anterior/medial corner, wherein the second suture is secured to the posterior/medial corner and the anterior/lateral corner such that a posterior/medial tail extends from the posterior/medial corner and an anterior/lateral tail extends from the anterior/lateral corner, and the first suture and the second suture intersect on the graft and retain the graft flat.

In an embodiment, the method includes retrieving the posterior/medial suture tail and then pulling the posterior/medial suture tail through an incision, retrieving the anterior/medial suture tail and then pulling the anterior/medial suture tail through an incision, retrieving the posterior/lateral suture tail and then pulling the posterior/lateral suture tail through an incision, and retrieving the anterior/lateral suture tail and then pulling the anterior/lateral suture tail through an incision.

In an embodiment, the method includes retrieving the posterior/lateral suture tail and the anterior/medial suture tail of the first suture through a lateral side through a medial percutaneous incision, tying the posterior/lateral suture tail and the anterior/medial suture tail in a first knot on the medial side of the acromion, retrieving the anterior/lateral suture tail and the posterior/medial suture tail of the second suture through the lateral side through the medial percutaneous incision, and tying the anterior/lateral suture tail and the posterior/medial suture tail in a second knot on the medial side of the acromion.

In an embodiment, the method includes trimming the posterior/lateral suture tail and the anterior/medial suture tail of the first knot and trimming the anterior/lateral suture tail and the posterior/medial suture tail of the second knot.

In an embodiment, the method includes including loading the posterior/medial suture tail and the anterior/medial suture tail into the introducer instrument, locating the graft under the acromion, retrieving the posterior/medial suture tail and the anterior/medial suture tail from portals, removing the introducer instrument, and retrieving the posterior/lateral suture tail and the anterior/lateral suture tail from portals

In an embodiment, the method includes wrapping the graft around the acromion, wherein the graft includes a body portion, a first end region, and a second end region including a slot, and inserting the first end region of the graft through the slot in the second end region of the graft and tensioning the graft around the acromion.

In an embodiment, the method includes stitching the first end region and the second end region together to secure the graft to the acromion.

Another embodiment of a method of resurfacing an acromion disclosed herein include attaching a first suture and a second suture to a graft, wherein the graft is substantially rectangular in shape and includes a posterior/medial corner, an anterior/medial corner, a posterior/lateral corner, and an anterior/lateral corner. The first suture is secured to the posterior/lateral corner and the anterior/medial corner such that a posterior/lateral tail extends from the posterior/lateral corner and an anterior/medial tail extends from the anterior/medial corner. The second suture is secured to the posterior/medial corner and the anterior/lateral corner such that a posterior/medial tail extends from the posterior/medial corner and an anterior/lateral tail extends from the anterior/lateral corner. The first suture and the second suture intersect on the graft and retain the graft flat. The method includes loading the first suture and the second suture into an introducer instrument that spreads the graft to be substantially flat, positioning the graft under the acromion, retrieving the posterior/medial suture tail and then pulling the posterior/medial suture tail through an incision, retrieving the anterior/medial suture tail and then pulling the anterior/medial suture tail through an incision, retrieving the posterior/lateral suture tail and then pulling the posterior/lateral suture tail through an incision, and retrieving the anterior/lateral suture tail and then pulling the anterior/lateral suture tail through an incision. The method includes retrieving the posterior/lateral suture tail and the anterior/medial suture tail of the first suture through a lateral side through a medial percutaneous incision, tying the posterior/lateral suture tail and the anterior/medial suture tail in a first knot on the medial side of the acromion, retrieving the anterior/lateral suture tail and the posterior/medial suture tail of the second suture through the lateral side through the medial percutaneous incision, and tying the anterior/lateral suture tail and the posterior/medial suture tail in a second knot on the medial side of the acromion. The method includes trimming the posterior/lateral suture tail and the anterior/medial suture tail of the first knot, and trimming the anterior/lateral suture tail and the posterior/medial suture tail of the second knot.

In an embodiment, the method includes measuring a surface area of the acromion and trimming the graft to fit the acromion based on a measurement.

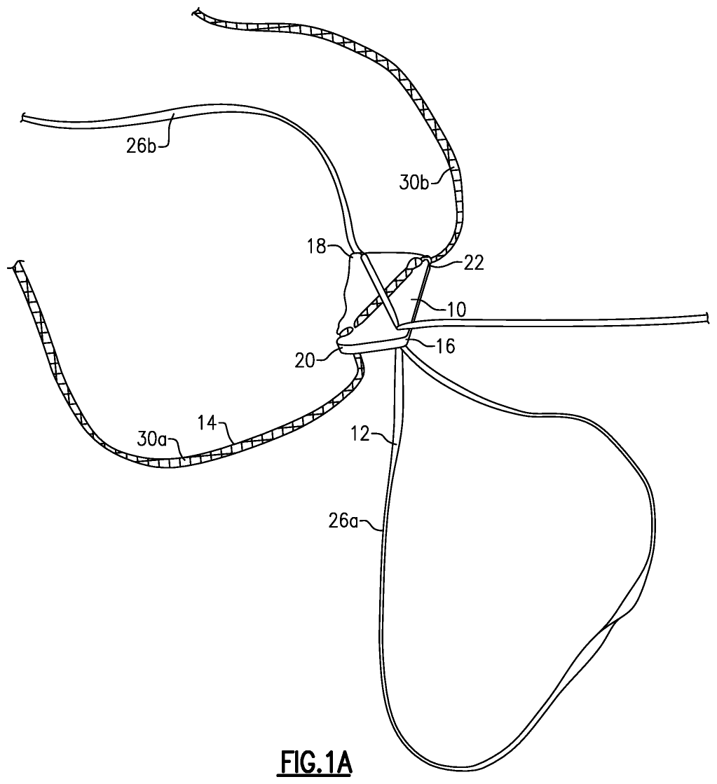

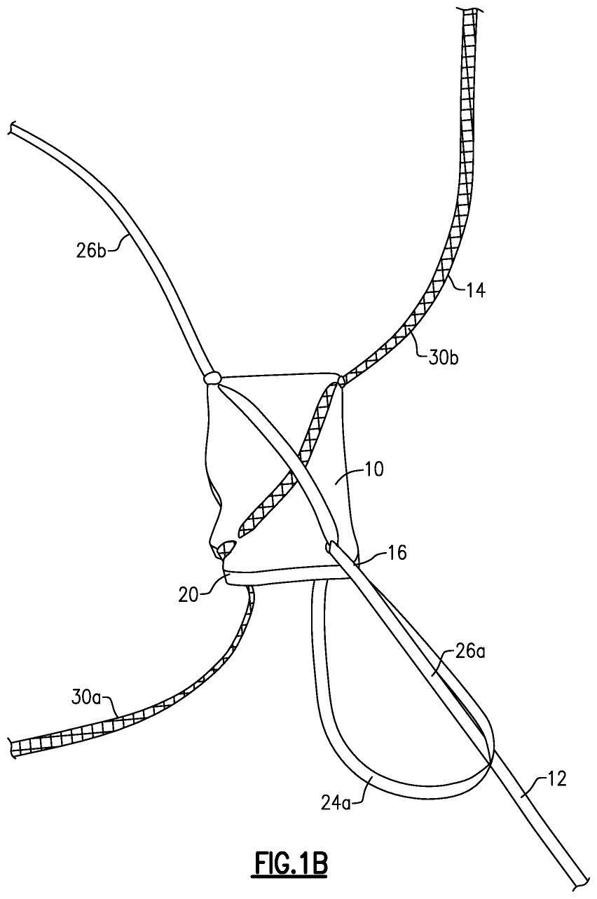

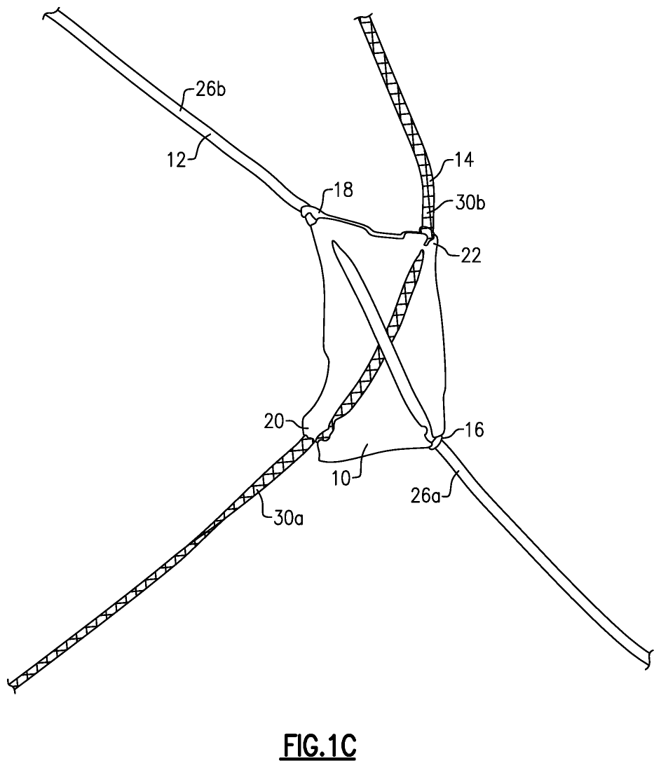

FIGS. 1A, 1B, and 1C illustrate preparation of a graft 10 with a first suture 12 and a second suture 14. A surface area of an acromion 36 of a scapula is measured, and the graft 10 is trimmed to an appropriate size. A skin marker (not shown) on the graft 10 can be used to help identify the orientation of the graft 10 during preparation and implantation.

The graft 10 is substantially rectangular in shape and includes four corners 16, 18, 20 and 22. A first suture 12 and a second suture 14 are pre-passed through the graft 10 outside of a body. A suture loop 24a of the first suture 12 is passed through a posterior/lateral corner 16 of the graft 10, and a posterior/lateral tail 26a of the first suture 12 is fed through the suture loop 24a and tightened to remove any slack (this technique is also called a “Lasso Loop”). Another suture loop (not shown) of the first suture 12 is passed through an anterior/medial corner 18 of the graft 10, and an anterior/medial tail 26b of the first suture 12 is fed through the suture loop (not shown) and tightened to remove any slack. A suture loop 28a of the second suture 14 is passed through an anterior/lateral corner 20 of the graft 10, and an anterior/lateral tail 30a of the second suture 14 is fed through the suture loop 28a and tightened to remove any slack. Another suture loop (not shown) of the second suture 14 is passed through a posterior/medial corner 22 of the graft 10, and a posterior/medial tail 30b of the second suture 14 is fed through the suture loop (not shown) and tightened to remove any slack. The first suture 12 and the second suture 14 intersect on the graft 10 in an X liguration with the tails 26a, 26b, 30a, and 30b extending from the corners 16, 18, 20, and 2, respectively.

The “Lasso Loop” technique secures the first suture 12 and the second suture 14 to the graft 10 and holds the graft 10 flat and spread wide open in a rectangular shape relative to the acromion 36. The attachment of the first suture 12 and the second suture 14 prevent sliding during delivery and fixation of the graft 10.

In another example, the two sutures 12 and 14 are suture tape. Although a specific suture configuration on the graft 10 has been illustrated and described, it is possible for other suture configurations to be used.

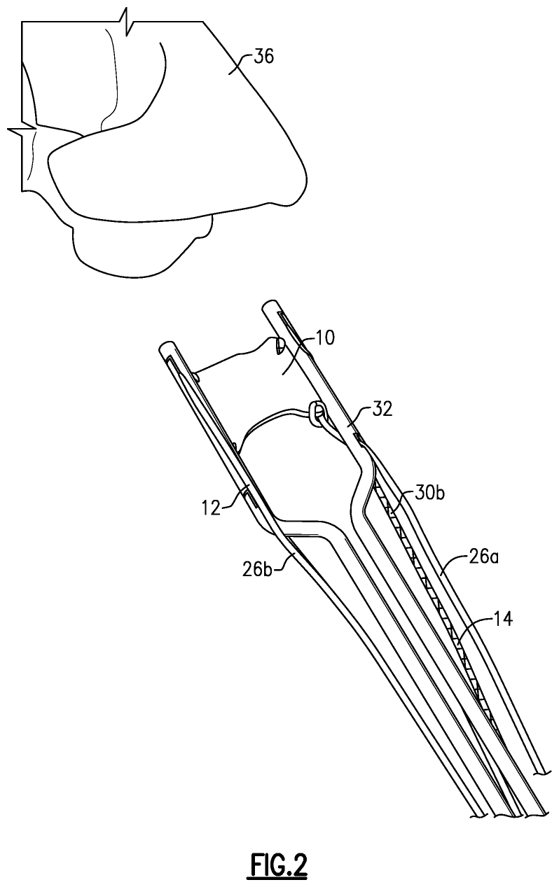

FIG. 2 illustrates the delivery of the graft 10 the acromion 36. The two sutures 12 and 14 are loaded into an introducer instrument 32, and the introducer instrument 32 spreads the graft 10 flat and locates the graft 10 under the acromion 36. Notches or cleats (not shown) on the introducer instrument 32 retain the sutures 12 and 14. The introducer instrument 32 comes through a lateral portal and spreads and locates the graft 10 under the acromion 36.

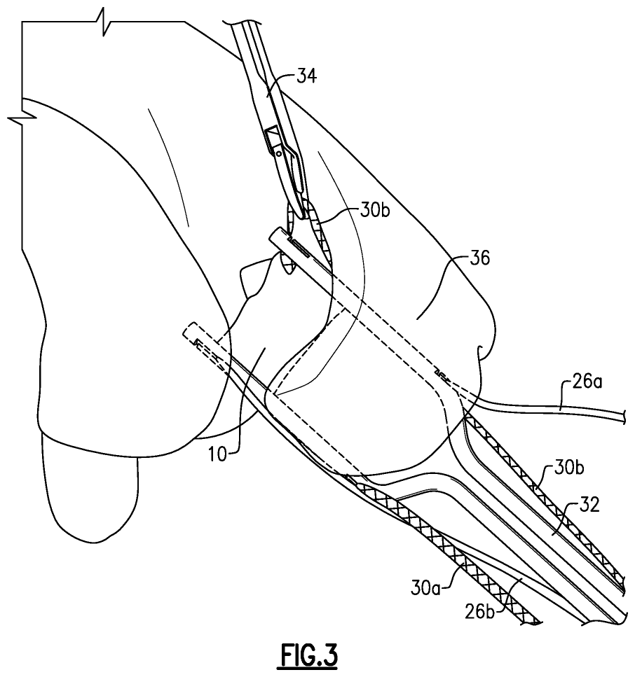

FIGS. 3, 4a, 4b, 5a, 5b, 6a, and 6b illustrate retrieval of the suture tails 26a, 26b, 30a, and 30b of the sutures 12 and 14. Four percutaneous incisions are created to retrieve the suture tails 26a, 26b, 30a, and 30b attached to the graft 10.

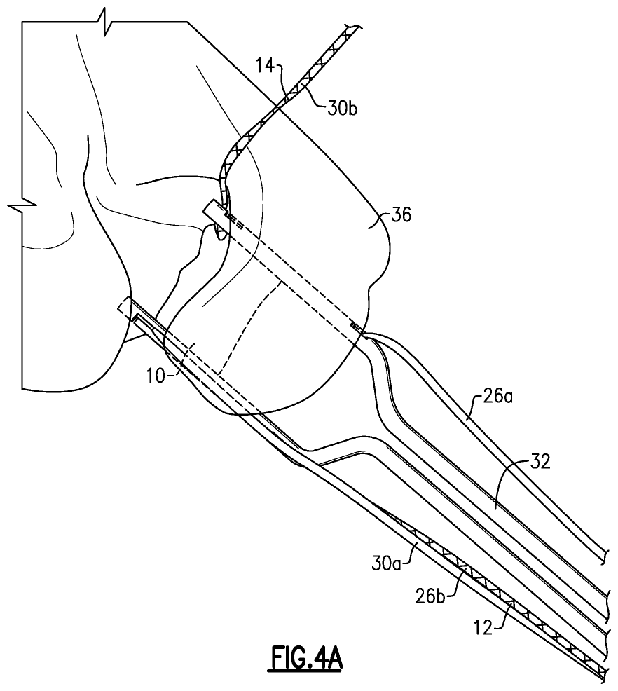

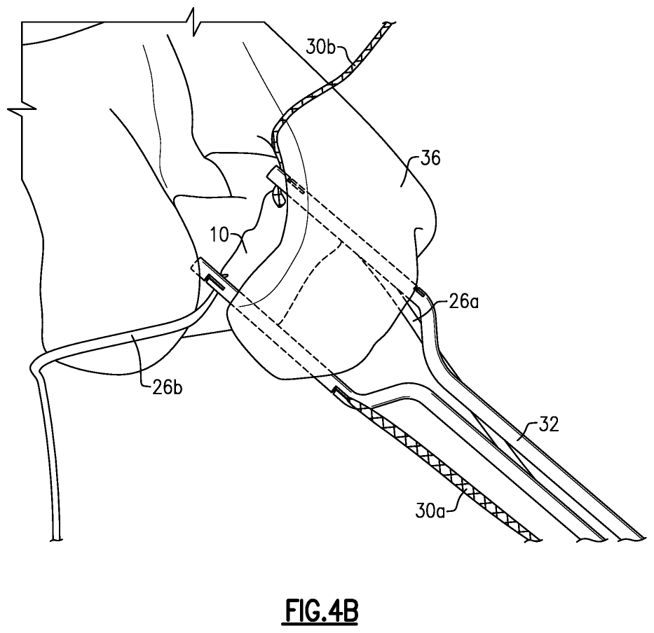

As shown in FIG. 3, a suture grasper 34 is used to grasp and retrieve the posterior/medial suture tail 30b. As shown in FIGS. 4a, the posterior/medial suture tail 30b is then pulled through the respective percutaneous incision. The suture grasper 34 is used to grasp and retrieve the anterior/medial suture tail 26b (not shown).

As shown in FIG. 4b, the anterior/medial suture tail 26b is then pulled through the respective percutaneous incision.

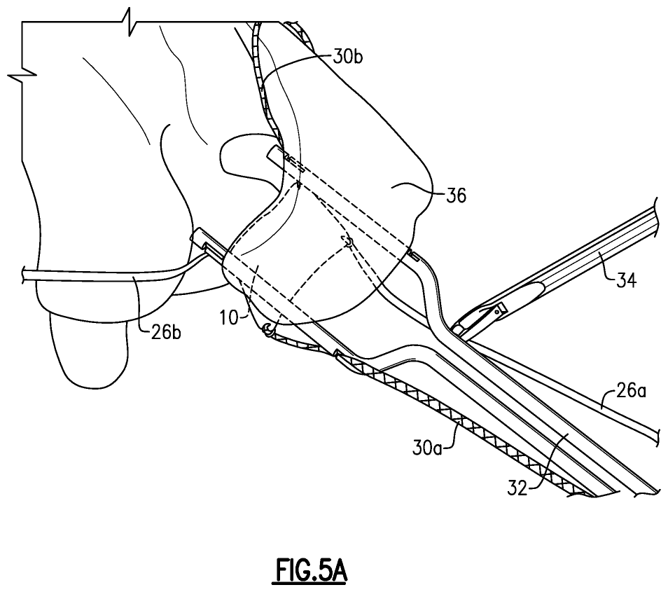

As shown in FIG. 5a, the suture grasper 34 is used to grasp and retrieve the posterior/lateral suture tail 26a. As shown in FIG. 5b, the posterior/lateral suture tail 26a is then pulled through the respective percutaneous incision.

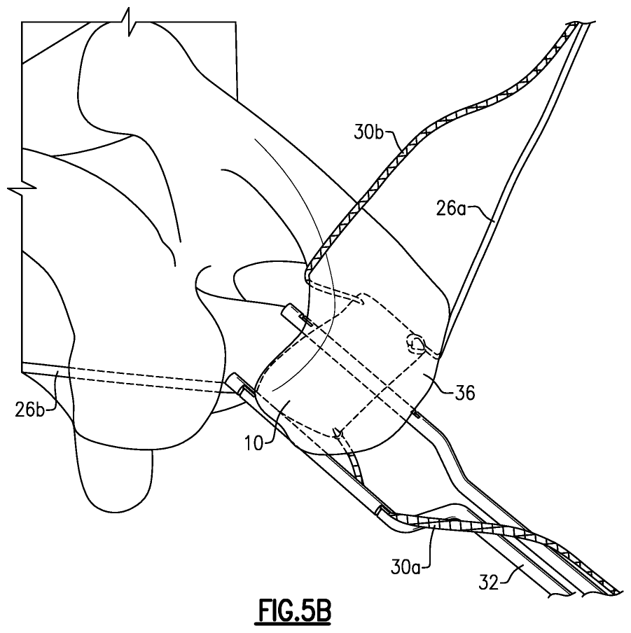

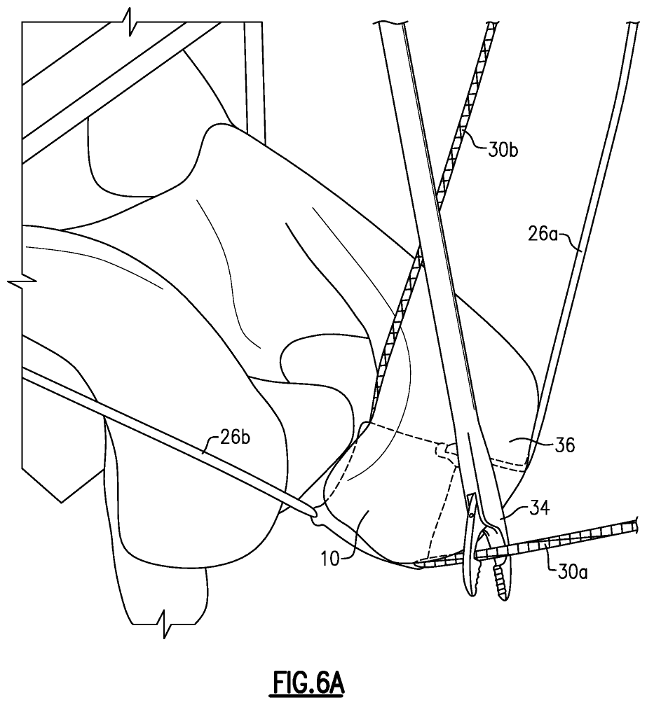

As shown in FIG. 6a, the suture grasper 34 is used to grasp and retrieve the anterior/lateral suture tail 30a. As shown in FIG. 6b, the anterior/lateral suture tail 30a is then pulled through the respective percutaneous incision.

In another example, the anterior/medial suture tail 26b and the posterior/medial suture tail 30b of the sutures 12 and 14, respectively, are loaded into the introducer instrument 32. The introducer instrument 32 is located under the acromion 36, and the anterior/medial suture tail 26b and the posterior/medial suture tail 30b are retrieved out of respective portals (not shown). The introducer instrument 32 is then removed from the body, and the posterior/lateral suture tail 26a and the anterior/lateral suture tail 30a are retrieved out of respective portals (not shown).

In another example, the graft 10 can be introduced under the acromion 36 without the introducer instrument 32, and the sutures 12 and 14 can be retrieved while the graft 10 is inside or outside the body.

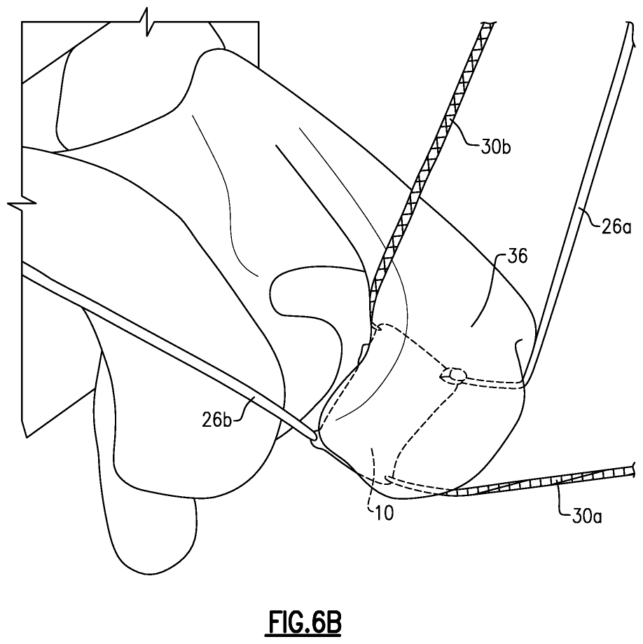

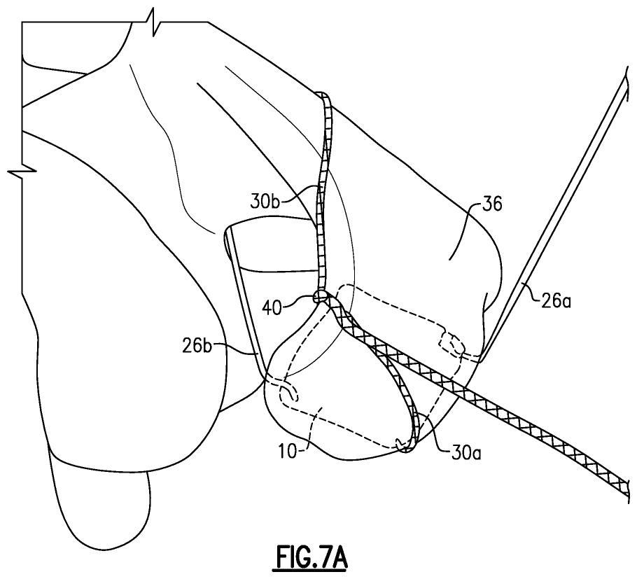

FIGS. 7a and 7b illustrate tying of the sutures 12 and 14 about the acromion 36 to secure the graft 10 to the acromion 36. The posterior/lateral suture tail 26a of the first suture 12 is retrieved from the lateral side through the medial percutaneous incisions and then tied to the anterior/medial suture tail 26b of the first suture 12 on a medial side of the acromion 36 to form a knot 38. The anterior/lateral suture tail 30a of the second suture 14 is retrieved from the lateral side through the medial percutaneous incisions and then tied to the posterior/medial suture tail 30b of the second suture 14 on the medial side of the acromion 36 to form a knot 40.

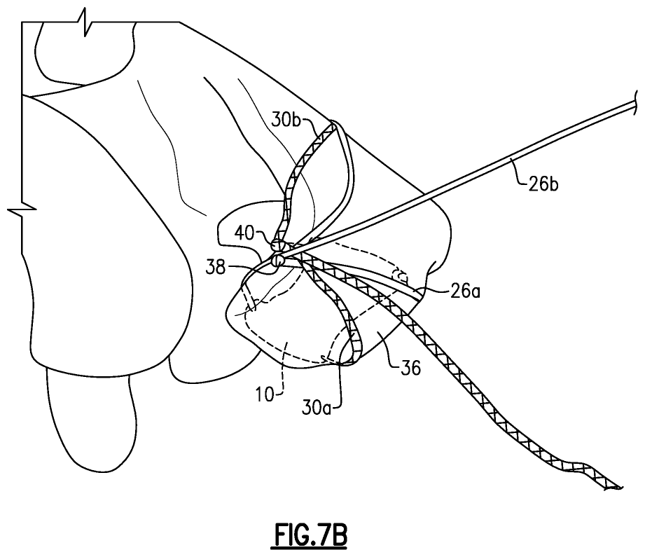

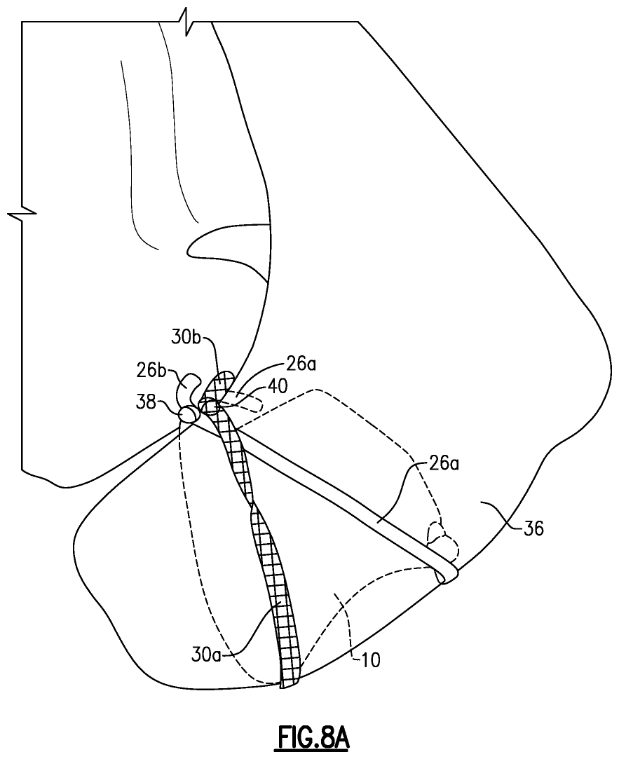

FIGS. 8a and 8b illustrates the graft 10 secured to the acromion 36 with the sutures 12 and 14 and the knots 38 and 40, respectively. The sutures 12 and 14 are trimmed after the knots 38 and 40, respectively, are tied, securing the graft 10 to the acromion 36.

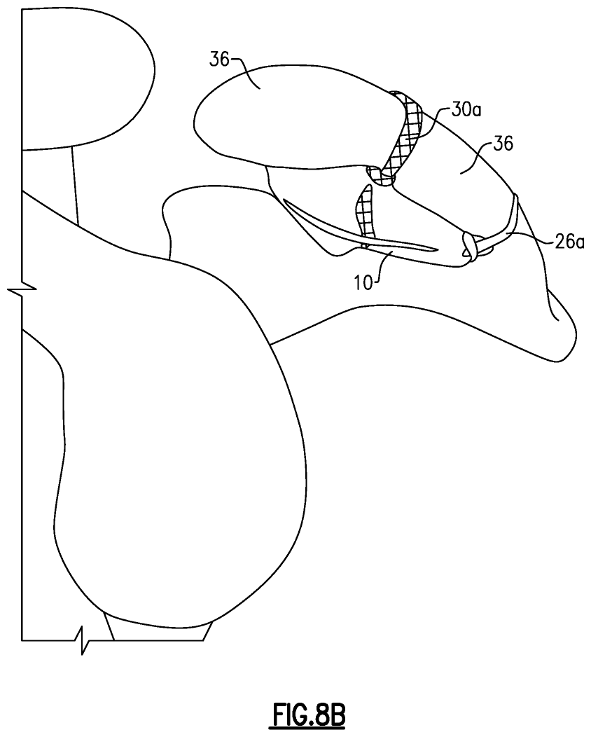

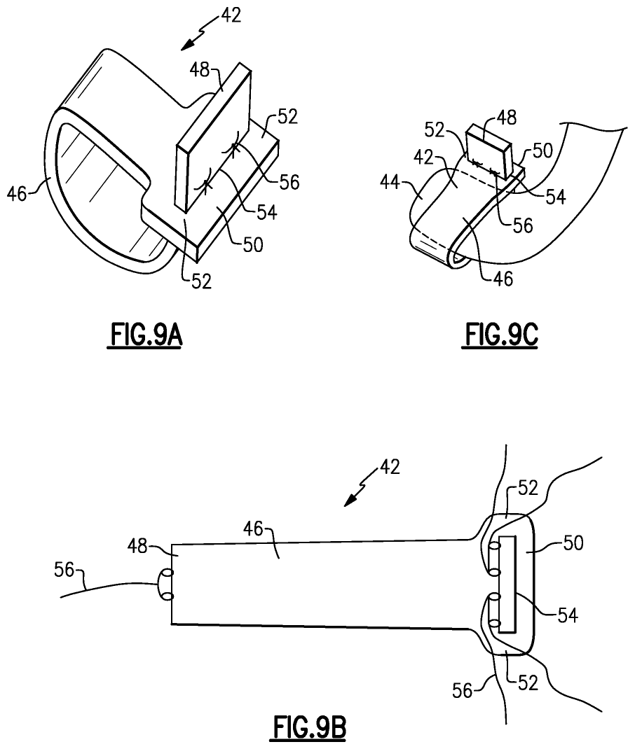

FIGS. 9a, 9b, and 9c illustrate another embodiment of a graft 42 attached to an acromion 44 like a “zip tie.” The graft 42 includes a body portion 46 having a first end region 48 and an opposing second end region 50. The second end region 50 includes two outwardly extending tabs 52 such that the second end region 50 is greater in width than the body portion 46. The second end region 48 also includes a slot 54.

The graft 42 is wrapped the around the acromion 44, as shown in FIG. 9c. The first end region 48 of the graft 42 is inserted through the slot 54 in the second end region 50 and tensioned around the acromion 44. Suture 56 is used to stitch the end portions 48 and 50 of the graft 46 together, and the suture 56 is then knotted to retain the graft 46 on the acromion 46.

When the humerus articulates against the acromion of the scapula because of a deficient rotator cuff, shoulder joint pain can occur. The graft attached to the acromion resurfaces the acromion and acts as a padding between the humerus and the acromion to reduce pain.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A method of resurfacing an acromion, comprising:
attaching at least one suture to a graft; and
securing the graft to an acromion with the at least one suture.

2. The method as recited in claim 1 including measuring a surface area of the acromion and trimming the graft to fit the acromion based on a measurement.

3. The method as recited in claim 1 wherein the at least one suture includes a first suture and a second suture, the method including loading the first suture and the second suture into an introducer instrument that spreads the graft to be substantially flat and positioning the graft under the acromion.

4. The method as recited in claim 3
wherein the graft is substantially rectangular in shape and includes a posterior/medial corner, an anterior/medial corner, a posterior/lateral corner, and an anterior/lateral corner,
wherein the first suture is secured to the posterior/lateral corner and the anterior/medial corner such that a posterior/lateral tail extends from the posterior/lateral corner and an anterior/medial tail extends from the anterior/medial corner,
wherein the second suture is secured to the posterior/medial corner and the anterior/lateral corner such that a posterior/medial tail extends from the posterior/medial corner and an anterior/lateral tail extends from the anterior/lateral corner, and
wherein the first suture and the second suture intersect on the graft and retain the graft flat.

5. The method as recited in claim 4 including retrieving the posterior/medial suture tail and then pulling the posterior/medial suture tail through an incision, retrieving the anterior/medial suture tail and then pulling the anterior/medial suture tail through an incision, retrieving the posterior/lateral suture tail and then pulling the posterior/lateral suture tail through an incision, and retrieving the anterior/lateral suture tail and then pulling the anterior/lateral suture tail through an incision.

6. The method as recited in claim 5 including retrieving the posterior/lateral suture tail and the anterior/medial suture tail of the first suture through a lateral side through a medial percutaneous incision, tying the posterior/lateral suture tail and the anterior/medial suture tail in a first knot on the medial side of the acromion, retrieving the anterior/lateral suture tail and the posterior/medial suture tail of the second suture through the lateral side through the medial percutaneous incision, and tying the anterior/lateral suture tail and the posterior/medial suture tail in a second knot on the medial side of the acromion.

7. The method as recited in claim 6 including trimming the posterior/lateral suture tail and the anterior/medial suture tail of the first knot and trimming the anterior/lateral suture tail and the posterior/medial suture tail of the second knot.

8. The method as recited in claim 4 including loading the posterior/medial suture tail and the anterior/medial suture tail into the introducer instrument, locating the graft under the acromion, retrieving the posterior/medial suture tail and the anterior/medial suture tail from portals, removing the introducer instrument, and retrieving the posterior/lateral suture tail and the anterior/lateral suture tail from portals.

9. The method as recited in claim 1 including wrapping the graft around the acromion, wherein the graft includes a body portion, a first end region, and a second end region including a slot, and inserting the first end region of the graft through the slot in the second end region, and tensioning the graft around the acromion.

10. The method as recited in claim 9 including stitching the first end region and the second end region together to secure the graft to the acromion.

11. A method of resurfacing an acromion, comprising:
attaching a first suture and a second suture to a graft, wherein the graft is substantially rectangular in shape and includes a posterior/medial corner, an anterior/medial corner, a posterior/lateral corner, and an anterior/lateral corner, wherein the first suture is secured to the posterior/lateral corner and the anterior/medial corner such that a posterior/lateral tail extends from the posterior/lateral corner and an anterior/medial tail extends from the anterior/medial corner, wherein the second suture is secured to the posterior/medial corner and the anterior/lateral corner such that a posterior/medial tail extends from the posterior/medial corner and an anterior/lateral tail extends from the anterior/lateral corner, and the first suture and the second suture intersect on the graft and retain the graft flat;
loading the first suture and the second suture into an introducer instrument that spreads the graft to be substantially flat;
positioning the graft under the acromion;
retrieving the posterior/medial suture tail and then pulling the posterior/medial suture tail through an incision;
retrieving the anterior/medial suture tail and then pulling the anterior/medial suture tail through an incision;
retrieving the posterior/lateral suture tail and then pulling the posterior/lateral suture tail through an incision;
retrieving the anterior/lateral suture tail and then pulling the anterior/lateral suture tail through an incision;
retrieving the posterior/lateral suture tail and the anterior/medial suture tail of the first suture through a lateral side through a medial percutaneous incision;
tying the posterior/lateral suture tail and the anterior/medial suture tail in a first knot on the medial side of the acromion;
retrieving the anterior/lateral suture tail and the posterior/medial suture tail of the second suture through the lateral side through the medial percutaneous incision;
tying the anterior/lateral suture tail and the posterior/medial suture tail in a second knot on the medial side of the acromion;
trimming the posterior/lateral suture tail and the anterior/medial suture tail of the first knot; and
trimming the anterior/lateral suture tail and the posterior/medial suture tail of the second knot.

12. The method as recited in claim 11 including measuring a surface area of the acromion and trimming the graft to fit the acromion based on a measurement.
